SEQUENCE LISTING (26 sequences)

SEQ ID NO: 1 - protein, 250 aa, human
  AT-19 scFv antibody
QVQLVQSGGGLVQPGGSLRLSCAASGFTFSNYAMSWVRQAPGKGLEWVSAISGNGGATYYADSMKGRFTISRDNSKNTLSLQLNSLRVEDTAVYYCAKSLIWGYDRGVDYWGQGTLVTVSAGGGGSGGGGSGGGGSGAPHVILTQPPSASGTPGQRVTISCSGGASNIGTNSVNWYQQLPGTAPKLLLYTNDHRPSGVPDRFSGKSGTSASLAISGLQAEDEGDYYCAAWDDTMNGHVVFGGGTKLTVLG

SEQ ID NO: 2 - DNA, 701 bp, human
  AT-19 scFv antibody
cctgagactctcctgtgcagcctctggattcacctttagcaactatgccatgagctgggtccgccaggctccagggaaggctggagtgggtctcagctattagtggtaatggtggtgccacatactacgcagactccatgaagggccggttcaccatctccagagacaattccaagaacacgctgtctctgcaattgaacagcctgagagtcgaggacacggccgtatattactgtgcgaaatccctaatatggggctacgatcggggggttgactactggggccagggaaccctggtcaccgtctcggccggtggcggtggcagcggcggtggtgggtccggtggcggcggatctggcgcgccacacgttatactgactcaaccgccctcagcgtctgggacccccggacagagggtcaccatttcttgttctggaggcgcctccaacatcggaactaattctgtaaactggtaccagcaactcccaggcacggcccccaaactcctcctctatactaatgatcaccggccctcaggtgtccctgaccggttctctggctctaagtctggcacctcagcctccctggccatcagtgggctccaggctgaggatgagggtgattattactgtgcggcatgggatgacacaatgaatggccatgtggtgttcggcggagggaccaagctgaccgtcctaggt

SEQ ID NO: 3 - protein, 121 aa, human
  AT-19 heavy chain
  CDR1 (27)..(35): CDR1
  CDR2 (47)..(66): CDR2
  CDR3 (70)..(121): CDR3
QVQLVQSGGGLVQPGGSLRLSCAASGFTFSNYAMSWVRQAPGKGLEWVSAISGNGGATYYADSMKGRFTISRDNSKNTLSLQLNSLRVEDTAVYYCAKSLIWGYDRGVDYWGQGTLVTVSA

SEQ ID NO: 4 - protein, 111 aa, human
  AT-19 light chain
  CDR1 (25)..(35)
  CDR2 (47)..(55)
  CDR3 (92)..(99)
HVILTQPPSASGTPGQRVTISCSGGASNIGTNSVNWYQQLPGTAPKLLLYTNDHRPSGVPDRFSGKSGTSASLAISGLQAEDEGDYYCAAWDDTMNGHVVFGGGTKLTVLG

SEQ ID NO: 5 - DNA, 311 bp, human
  AT-19 heavy chain
cctgagactctcctgtgcagcctctggattcacctttagcaactatgccatgagctgggtccgccaggctccagggaaggctggagtgggtctcagctattagtggtaatggtggtgccacatactacgcagactccatgaagggccggttcaccatctccagagacaattccaagaacacgctgtctctgcaattgaacagcctgagagtcgaggacacggccgtatattactgtgcgaaatccctaatatggggctacgatcggggggttgactactggggccagggaaccctggtcaccgtctcggcc

SEQ ID NO: 6 - DNA, 337 bp, human
  AT-19 light chain
acacgttatactgactcaaccgccctcagcgtctgggacccccggacagagggtcaccatttcttgttctggaggcgcctccaacatcggaactaattctgtaaactggtaccagcaactcccaggcacggcccccaaactcctcctctatactaatgatcaccggccctcaggtgtccctgaccggttctctggctctaagtctggcacctcagcctccctggccatcagtgggctccaggctgaggatgagggtgattattactgtgcggcatgggatgacacaatgaatggccatgtggtgttcggcggagggaccaagctgaccgtcctaggt

SEQ ID NO: 7 - protein, 9 aa, homo sapien
  AT-19 heavy chain CDR1
FTFSNYAMS

SEQ ID NO: 8 - protein, 20 aa, homo sapien
  AT-19  heavy chain CDR2
WVSAISGNGGATYYADSMKG

SEQ ID NO: 9 - protein, 52 aa, homo sapien
  AT-19 heavy chain CDR3
ISRDNSKNTLSLQLNSLRVEDTAVYYCAKSLIWGYDRGVDYWGQGTLVTVSA

SEQ ID NO: 10 - protein, 13 aa, homo sapien
  AT-19 light chain CDR1
SGGASNIGTNSVN

SEQ ID NO: 11 - protein, 9 aa, homo sapien
  AT-19 light chain CDR2
LLLYTNDHR

SEQ ID NO: 12 - protein, 11 aa, homo sapien
  AT-19 light chain CDR3
AAWDDTMNGHV

SEQ ID NO: 13 - DNA, 27 bp, human
  AT-19 heavy chain CDR1
ttcacctttagcaactatgccatgagc

SEQ ID NO: 14 - DNA, 60 bp, human
  AT-19 heavy chain CDR2
tgggtctcagctattagtggtaatggtggtgccacatactacgcagactccatgaagggc

SEQ ID NO: 15 - DNA, 36 bp, human
  AT-19 heavy chain CDR3
tccctaatatggggctacgatcggggggttgactac

SEQ ID NO: 16 - DNA, 39 bp, human
  AT-19 light chain CDR1
tctggaggcgcctccaacatcggaactaattctgtaaac

SEQ ID NO: 17 - DNA, 27 bp, human
  AT-19 light chain CDR2
ctcctcctctatactaatgatcaccgg

SEQ ID NO: 18 - DNA, 32 bp, human
  AT-19 light chain CDR3
cggcatgggatgacacaatgaatggccatgtg

SEQ ID NO: 19 - DNA, 61 bp, artificial
  F-ss-VL cloning primer
caggaagcttgccaccatggaaaccccagcgcagcttctcttcctcctgctactctggctc

SEQ ID NO: 20 - DNA, 63 bp, artificial
  R-ss-VL cloning primer
agtcagtataacgtgactagttccggtggtatctgggagccagagtagcaggaggaagagaag

SEQ ID NO: 21 - DNA, 32 bp, artificial
  cloning primer F-VL
actagtcacgttatactgactcaaccgccctc

SEQ ID NO: 22 - DNA, 34 bp, artificial
  cloning primer R-VL
cgtgcgtacgacctaggacggtcagcttggtccc

SEQ ID NO: 23 - DNA, 59 bp, artificial
  cloning primer F-ss-VH
ataagaatgcggccgccaccatggagtttgtgctgagctgggttttccttgttgctata

SEQ ID NO: 24 - DNA, 70 bp, artificial
  cloning primer R-ss-VH
ctgcaccagctgcacctggatatcacactggacaccttttaatatagcaacaaggaaaacccagctcagc

SEQ ID NO: 25 - DNA, 29 bp, artificial
  cloning primer F-VH
gatatccaggtgcagctggtgcagtctgg

SEQ ID NO: 26 - DNA, 31 bp, artificial
  cloning primer R-VH
ctgccgctagccgagacggtgaccagggttc

(19) United States
(12) Patent Application Publication
(10) Pub. No.: US 2006/0292074 A1
(43) Pub. Date: Dec. 28, 2006

(54) Tomoregulin antibodies and uses thereof

(75) Inventors: Tara Heitner, Copenhagen V (DK); David Light, San Mateo, CA (US); Bing Liu, Richmond, CA (US); Noboru Satozawa, Richmond, CA (US); Xiao-Yan Zhao, Union City, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 11/473,444

(22) Filed: Jun. 23, 2006

Related U.S. Application Data
(60) Provisional application No. 60/695,138, filed on Jun. 28, 2005.

(51) Int. Cl. A61K 51/00; G01N 33/574; A61K 39/395; C07K 16/46; C07K 16/30

(52) U.S. Cl. 424/1.49; 530/388.8; 530/391.1; 435/7.23; 424/155.1; 424/178.1

(57) ABSTRACT

The present invention relates to antibodies, and antigen-binding antibody fragments, directed against a Tomoregulin (TR) polypeptide. The invention further relates to methods for utilizing the antibodies, and antibody fragments, for diagnostic and therapeutic applications.

This application claims the benefit of U.S. Provisional Application Ser. No. 60/695,138, filed on Jun. 28, 2005, which is incorporated herein by reference.

### BACKGROUND OF THE INVENTION

Antibody-based therapy is proving very effective in the treatment of various cancers. For example, HERCEPTIN® has been used successfully to treat breast cancer. Central to the development of a successful antibody-based therapy is isolation of antibodies against cell-surface proteins found to be preferentially expressed on tumor cells.

### FIELD OF THE INVENTION

This invention relates to novel antibodies directed against a cell-surface polypeptide, tomoregulin (TR), which is preferentially expressed in some cancer cells, particularly prostate tumor cells. The invention further relates to the use of these antibodies for the treatment and detection of cancer and cancer metastasis.

### SUMMARY OF THE INVENTION

The present invention provides human antibodies, or antigen-binding antibody fragments thereof, or variants thereof, that are highly selective for tomoregulin (TR), and which may be employed in methods for detection of TR expression, which is associated with disease states such as cancer of the prostate, and in the treatment of such disease states.

Toward these ends, it is an object of the invention to provide isolated human antibodies, or antigen binding antibody fragments thereof, that specifically bind to an epitope present in a TR polypeptide (SEQ ID NO:1). Particularly preferred are human antibodies that bind to an epitope of the TR polypeptide with a dissociation constant (K_{D}) which is less than or equal to 1 μM, more preferably less than or equal to 100 nM and most preferably less than or equal to 10 nM. Particularly preferred are antibody fragments selected from the group consisting of Fv, F(ab′), F(ab′)2, scFv, minibodies and diabodies.

In a preferred embodiment, the antibodies of the invention are internalized following binding to a TR expressing cell.

A preferred antibody of the invention comprises a heavy chain variable region having CDR1, CDR2 and CDR 3 regions comprising the amino acid sequences set forth in SEQ ID NOS:7, 8 and 9, respectively. Other preferred antibodies are antibodies comprising a light chain variable region having CDR1, CDR2 and CDR3 regions comprising the amino acid sequences set forth in SEQ ID NOS:13, 14 and 15, respectively. Particularly preferred is an antibody of the invention comprising a heavy chain variable region comprising CDR1, CDR2 and CDR3 regions comprising SEQ ID NOS:7, 8 and 9, repectively, and a light chain variable region having CDR1, CDR2 and CDR3 regions comprising the amino acid sequences set forth in SEQ ID NOS:13, 14 and 15, respectively.

Also provided are isolated antibodies and antigen-binding antibody fragments thereof, comprising a light chain variable region comprising the amino acid sequence of SEQ ID NO:4, a heavy chain variable region comprising the amino acid sequence of SEQ ID NO:3 and, particularly preferred, an isolated antibody comprising a light chain comprising the amino acid sequence of SEQ ID NO:4 and a heavy chain variable region comprising a heavy chain variable region comprising the amino acid sequence SEQ ID NO:3.

Particularly preferred is an anti-TR antibody having the amino acid sequence of SEQ ID NO:1.

Also provided are nucleic acid sequences which encode the light and heavy chain variable regions of the antibodies described above. Preferred is an antibody comprising a light chain variable region encoded by a nucleotide sequence comprising SEQ ID NO:6. Also preferred is an antibody comprising a heavy chain variable region encoded by a nucleotide sequence comprising SEQ ID NO:5. Particularly preferred, is an antibody encoded by the nucleotide sequence of SEQ ID NO:2.

In a further aspect of the invention, there are provided antibodies that are conjugated to a therapeutic agent, e.g. ricin or a radioisotope, for administration to cells in vitro, ex vivo and in vivo, or to a multicellular organism. Preferred in this regard are therapeutic agents that are cytotoxic.

In a further aspect of the invention is a method for treatment of a human patient of a disease state characterized by TR expression, such as prostate cancer, using the immunoconjugates of the invention.

Also provided are anti-TR antibodies conjugated to a detectable marker. Preferred markers are are a radiolabel, an enzyme, a chromophore or a fluorescer.

In a further aspect of the invention is a method for detection of a disease-state associated with TR expression, which uses the immunoconjugates of the present invention.

### BRIEF DESCRIPTION OF THE DRAWINGS

FIG. 1—Measurement of AT-19 scFv binding. FIG. 1, panel A, live cell ELISA with AT-19 scFv on TR-expressing PC3-TR clone 69. Binding of purified monovalent AT-19 scFv to native TR in a standard cell based ELISA is similar to non-specific binding by a control scFv at all concentrations tested (5 μg/mL=200 nM; 1 μg/mL=40 nM; 0.2 μg/mL=8 nM). The scFv's and secondary antibody were added sequentially for the ELISA. In contrast, FIG. 1, panel B, live cell ICELISA shows specific binding of AT-19 scFv after dimerization to native TR on PC3-TR cells. In the ICELISA, scFv were pre-incubated with mouse anti-E-tag monoclonal IgG to form an immune complex comprised of the anti-E-tag IgG molecule bound to two scFv. Non-specific binding by the immune complex of control scFv is low. In both the ELISA and ICELISA, Mab 2H8 is a positive anti-TR mouse IgG control that binds strongly in the ELISA, but poorly as the immune complex. Average of triplicate assays±SD.

FIG. 2—Concentration dependence of AT-19 scFv immune complex binding to native TR in the live cell ICELISA. AT-19 scFv was complexed with the secondary mouse anti-E-tag monoclonal antibody before addition to cells. The EC₅₀ of −8.5 nM is based upon the total concentration of scFv and is an underestimate of the potency of the dimer because the concentration of the immune complex is not known. All dilutions in triplicate.

FIG. 3—Binding of monomeric and dimerized AT-19 scFv to recombinant TR. Monomeric AT-19 scFv (-□-) binds recombinant TR with an EC₅₀ of 22 nM. The binding avidity increases 20-fold (EC₅₀ of 1.3 nM) when the scFv is dimerized by pre-incubation with the secondary antibody in an ICELISA (-◯-). Dilutions were performed in duplicate for the ELISA and in triplicate for the ICELISA.

FIG. 4—Binding of AT-19 IgG to PC3-TR cells. AT-19 antibody was expressed and purified as the fully human IgG (see Example 7) to create a stable dimeric antibody format. Binding of AT-19 IgG to PC3-TR cells was determined in a live cell ELISA using PC3-TR cells. The observed EC₅₀ of 0.44 nM can be compared to the lack of binding observed for monomeric AT-19 scFv from 8 to 200 nM (FIG. 1, panel A). All dilutions were performed in triplicate.

FIG. 5A-B—FACS analysis of binding of IgG molecules to PC3-TR cells. FIG. 5A, FACS analysis of binding of fully human AT-19 IgG (-◯-) to PC3-TR cells. Human IgG (-Δ-) is used as a control.

FIG. 5B, FACS analysis of mouse 2H8 IgG (-◯-) to PC3-TR cells. Murine IgG (-Δ-) is used as a control. The EC₅₀ for AT-19 IgG (0.17 nM) is similar to the EC₅₀ measured in the live cell ELISA (FIG. 4) and is within the same magnitude as the EC₅₀ for mouse anti-TR IgG 2H8 (0.67 nM).

FIG. 6—Amino acid sequence of AT-19 scFv. Amino acid sequence of the single chain of anti-TR antibody, AT-19 (SEQ ID NO:1), showing the VH and VL regions, the linker and the CDR regions.

FIG. 7—Nucleic acid sequence of AT-19 scFv. Nucleic acid sequence of the single chain of anti-TR antibody, AT-19 (SEQ ID NO:2). The CDR regions are underlined and the linker region is in italics.

### DETAILED DESCRIPTION OF THE INVENTION

Definitions

As used in the specification, examples and appended claims, unless specified to the contrary, the following terms have the meaning indicated.

A polypeptide “fragment”, “portion”, or “segment” is a stretch of amino acid residues of at least about 5 amino acids, often at least about 7 amino acids, typically at least about 9 to 13 amino acids, and in various embodiments, at least about 17 or more amino acids. “Fragment” refers to a polypeptide having an amino acid sequence that is entirely the same as part, but not all, of the amino acid sequence of the aforementioned TR polypeptides, or antibodies to TR, and variants or derivatives thereof.

“Deletion” is defined as a change in either polynucleotide or amino acid sequences in which one or more polynucleotides or amino acid residues, respectively, are absent.

“Insertion” or “addition” is that change in a polynucleotide or amino acid sequence which has resulted in the addition of one or more polynucleotides or amino acid residues, respectively, as compared to the naturally occurring polynucleotide or amino acid sequence.

“Substitution” results from the replacement of one or more polynucleotides or amino acids by different polynucleotides or amino acids, respectively.

“Variant(s)” of polynucleotides or polypeptides, as the term is used herein, are described below and elsewhere in the present disclosure in greater detail.

A variant of a polynucleotide is a polynucleotide that differs in polynucleotide sequence from another, reference polynucleotide. Generally, differences are limited so that the polynucleotide sequences of the reference and the variant are closely similar overall and, in many regions, identical.

Changes in the polynucleotide sequence of the variant may be silent. That is, they may not alter the amino acids encoded by the polynucleotide. Where alterations are limited to silent changes of this type a variant will encode a polypeptide with the same amino acid sequence as the reference. Also as noted below, changes in the polynucleotide sequence of the variant may alter the amino acid sequence of a polypeptide encoded by the reference polynucleotide. Such polynucleotide changes may result in amino acid substitutions, additions, deletions, fusions and truncations in the polypeptide encoded by the reference sequence, as discussed below.

A variant of a polypeptide is a polypeptide that differs in amino acid sequence from another, reference polypeptide. Generally, differences are limited so that the sequences of the reference and the variant are closely similar overall and, in many regions, identical. A variant and reference polypeptide may differ in amino acid sequence by one or more substitutions, additions, deletions, fusions and truncations, which may be present in any combination. Recombinant variants encoding these same or similar polypeptides may be synthesized or selected by making use of the “redundancy” in the genetic code. Various codon substitutions, such as the silent changes that produce various restriction sites, may be introduced to optimize cloning into a plasmid or viral vector or expression in a particular prokaryotic or eukaryotic system. Mutations may also be introduced to modify the properties of the polypeptide, to change ligand-binding affinities, interchain affinities, or polypeptide degradation or turnover rate.

As discussed herein, minor variations in the amino acid sequences of polypeptides, antibodies or immunoglobulin molecules are contemplated as being encompassed by the present invention, providing that the variations in the amino acid sequence maintain at least 80%, more preferably at least 85%, 90%, 95%, and most preferably 99% of the original sequence. In particular, conservative amino acid replacements are contemplated. Conservative replacements are those that take place within a family of amino acids that are related in their side chains. Genetically encoded amino acids are generally divided into families: (1) acidic (aspartate, glutamate); (2) basic (lysine, arginine, histidine); (3) non-polar (alanine, valine, leucine, isoleucine, proline, phenylalanine, methionine, tryptophan); and (4) uncharged polar (glycine, asparagine, glutamine, cysteine, serine, threonine, tyrosine). More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by comparing the specific activity of the polypeptide derivative with the unmodified polypeptide. For purposes of this application, the invention encompasses variants of the claimed antibodies which maintain a binding affinity (K_{D}) less than 1 μM for an TR epitope.

The following terms are used to describe the sequence relationships between two or more polynucleotide or amino acid sequences: “reference sequence”, “comparison window”, “sequence identity”, “percentage of sequence identity”, “substantial identity”, “similarity”, and “homologous”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence. The term “similarity”, when used to describe a polypeptide, is determined by comparing the amino acid sequence and the conserved amino acid substitutes of one polypeptide to the sequence of a second polypeptide. The term “homologous”, when used to describe a polynucleotide, indicates that two polynucleotides, or designated sequences thereof, when optimally aligned and compared, are identical, with appropriate nucleotide insertions or deletions, in at least 70% of the nucleotides, usually from about 75% to 99%, and more preferably at least about 98 to 99% of the nucleotides.

“Antibody” or “antigen-binding antibody fragment” refers to an intact antibody, or a fragment thereof, that competes with the intact antibody for specific binding. An antibody or antigen-binding antibody fragment, is said to specifically bind an antigen when the dissociation constant is less than or equal to 1 μM, preferably less than or equal to 100 nM and most preferably less than or equal to 10 nM. Binding can be measured by methods known to those skilled in the art. Binding to the native antigen expressed on a cell surface may be defined as the concentration of antibody or antibody fragment required to obtain one half of the maximal signal (EC₅₀) in an antibody titration experiment. Binding to cells may be determined in a live cell or fixed cell ELISA (enzyme-linked immunosorbant assay) or by FACS (fluorescence-activated cell sorter) analysis. Antibody fragments comprise a portion of an intact antibody, preferably the antigen binding or variable region of the intact antibody. Binding fragments include Fab, Fab′, F(ab′)₂, and Fv fragments; diabodies; linear antibodies; single-chain antibody molecules (scFv); and multispecific antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “bispecific” or “bifunctional” antibody is understood to have each of its binding sites identical.

“Epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. Two antibodies are said to “bind the same epitope” if one antibody is shown to compete with the second antibody in a competitive binding assay, by any of the methods well known to those of skill in the art.

“Recombinant” or “recombinant DNA molecule” refers to a polynucleotide sequence which is not naturally occurring, or is made by the artificial combination of two otherwise separated segments of sequence. By “recombinantly produced” is meant artificial combination often accomplished by either chemical synthesis means, or by the artificial manipulation of isolated segments of polynucleotides, e.g., by genetic engineering techniques. Such manipulation is usually done to replace a codon with a redundant codon encoding the same or a conservative amino acid, while typically introducing or removing a sequence recognition site. Alternatively, it is performed to join together polynucleotide segments with desired functions to generate a single genetic entity comprising a desired combination of functions not found in the common natural forms. Restriction enzyme recognition sites, regulation sequences, control sequences, or other useful features may be incorporated by design. “Recombinant DNA molecules” include cloning and expression vectors. “Recombinant” may also refer to a polynucleotide which encodes a polypeptide and is prepared using recombinant DNA techniques.

“Isolated” means altered “by the hand of man” from its natural state; i.e., that, if it occurs in nature, it has been changed or removed from its original environment, or both. For example, a naturally occurring polynucleotide or a polypeptide naturally present in a living animal in its natural state is not “isolated”, but the same polynucleotide or polypeptide separated from the coexisting materials of its natural state is “isolated”, as the term is employed herein. For example, with respect to polynucleotides, the term isolated means that it is separated from the chromosome and cell in which it naturally occurs. Polynucleotides and polypeptides may occur in a composition, such as media formulations, solutions for introduction of polynucleotides or polypeptides, for example, into cells, compositions or solutions for chemical or enzymatic reactions, for instance, which are not naturally occurring compositions, and, therein remain isolated polynucleotides or polypeptides within the meaning of that term as it is employed herein.

“Therapeutically effective dose” refers to that amount of polypeptide or its antibodies, antagonists, or inhibitors, including antisense molecules and ribozymes, which ameliorate the symptoms or conditions of a disease state. A dose is considered a therapeutically effective dose in the treatment of cancer or its metastasis when tumor or metastatic growth is slowed or stopped, or the tumor or metastasis is found to shrink in size, so as to lead to an extension in life span for the subject. A dose is also considered therapeutically effective if it leads to an improvement in the overall quality of life of the patient, i.e. alleviation of pain. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀.

“Treating” or “treatment” as used herein covers the treatment of a disease-state in a human patient, which disease-state includes disease states which are characterized by an increased level of TR, such as prostate cancer or advanced metastatic prostate cancer.

### DETAILED DESCRIPTION OF THE INVENTION

Antibodies

The present invention relates to human monoclonal antibodies, antigen-binding antibody fragments thereof, and variants of the antibodies and fragments, that specifically bind to a TR polypeptide (Uchida et al. Biochem. Biophys. Res. Comm. (1999) 266:593-602). Particularly preferred are antibodies, antigen-binding antibody fragments thereof, and variants of the antibodies and fragments that are internalized after binding to cells expressing TR. The methods described herein, however, are useful for the isolation of a human monoclonal antibody directed against any cell-surface antigen of interest.

The antibodies, antigen-binding antibody fragments, and variants of the antibodies and fragments of the invention are comprised of a light chain variable region and a heavy chain variable region. Variants of the antibodies or antigen-binding antibody fragments contemplated in the invention are molecules in which the binding activity of the antibody or antigen-binding antibody fragment for TR is maintained.

One embodiment of the invention are antibodies, or antigen-binding antibody fragments thereof, comprising a light chain variable region with CDR1, CDR2 and CDR3 regions comprising SEQ ID NOS 16, 17 and 18, respectively (corresponding to amino acid residues 25-35, 47-55 and 92-99 of SEQ ID NO:4) and a heavy chain variable region with CDR1, CDR2 and CDR3 regions comprising SEQ ID NOS:13, 14 and 15, respectively (corresponding to amino acid residues 27-35, 47-66 and 70-121 of SEQ ID NO: 3).

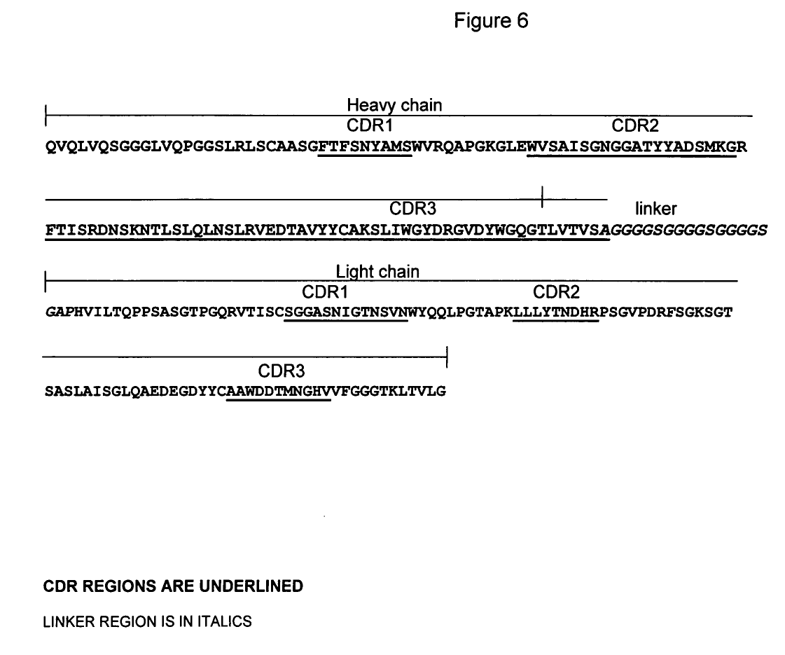

Among the preferred embodiments of the invention in this regard are antibodies, antigen-binding antibody fragments thereof, or variants thereof, comprising a light chain variable region having at least 80%, more preferrably at least 90%, still more preferrably at least 95%, and still more preferrably 99% sequence identity to SEQ ID NO:4 (amino acid residues 140-250 of SEQ ID NO:1, FIG. 6). Also preferred embodiments are antibodies, antigen-binding antibody fragments thereof, or variants thereof, comprising a heavy chain variable region having at least 80%, more preferrably at least 90%, still more preferrably at least 95%, and still more preferrably 99% sequence identity to SEQ ID NO:3 (amino acid residues 1-121 of SEQ ID NO:1, FIG. 6).

A preferred embodiment of the invention is an antibody, or antigen-binding antibody fragment thereof, or variants thereof, comprising a light chain variable region comprising the amino acid sequence of SEQ ID NO:4. Another preferred embodiment is an antibody, or antigen-binding antibody fragment thereof, or variants thereof, comprising a heavy chain variable region comprising the amino acid sequence of SEQ ID NO:3. A most preferred embodiment is an antibody, or antigen-binding antibody fragment thereof, or variants thereof, comprising a light chain variable region comprising the amino acid sequence of SEQ ID NO:4 and a heavy chain variable region comprising the amino acid sequence of SEQ ID NO:3.

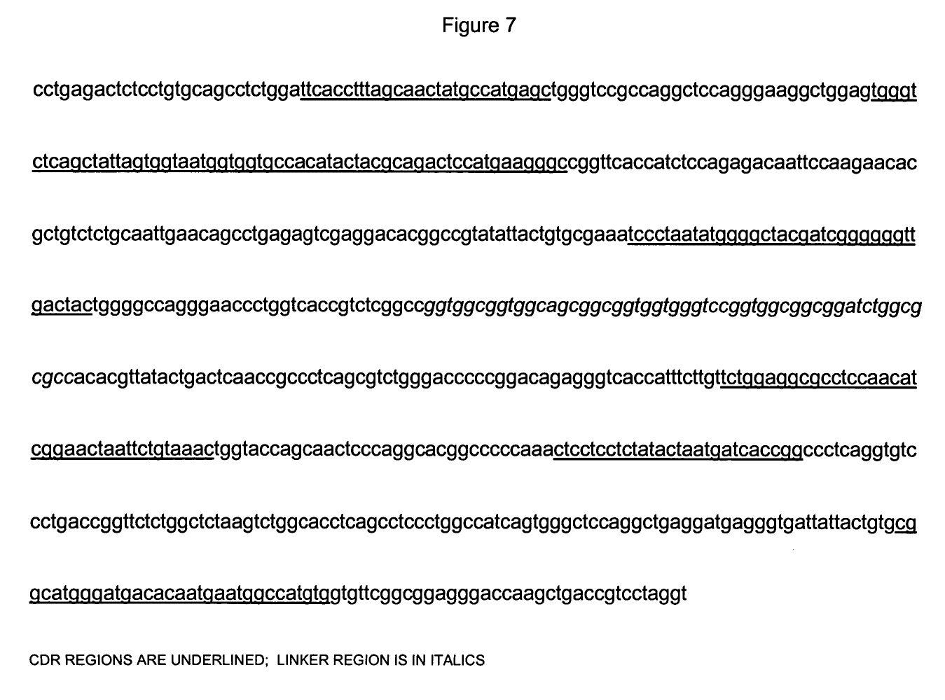

Particularly preferred is an antibody, or antigen-binding antibody fragments thereof, or variants thereof, comprising the amino acid sequence of SEQ ID NO:1 (FIG. 6), which is encoded by the nucleotide sequence of SEQ ID NO:2 (FIG. 7).

Antibody Production

A human, naïve antibody phage display library was used to isolate a high affinity, TR-specific, human monoclonal antibody, AT-19, by a combination of alternative panning and screening methods and through the development of specific tools. These tools and methods include a multivalent human scFv phage display library, the creation of two TR-expressing recombinant cell-lines and the development of a screening assay capable of identifying antibodies that preferentially bind in a bivalent format.

An antibody to the prostate cancer cell-surface marker, tomoregulin (TR), was discovered by a combination of three non-conventional approaches in phage-display technology (PDT). First, a multivalent scFv phage display library was generated by rescuing the stock library and each round of panning with hyperphage. Second, dual-alternating cell-surface selections were employed with cell-lines derived from two species (hamster and human) and pre-adsorption with appropriate control cells was included in each round of panning. Third, scFv were screened using an immunocomplex ELISA format to allow mutivalent antibody binding. The combination of these specific methods allowed the isolation of the unique antibody, AT-19.

Identification of antibodies to TR using phage display technology appears to be very sensitive to the method of antigen presentation. Standard selection strategies initially failed to yield antibodies that recognize native TR on cells. These initial strategies were similar to many literature reports where panning on recombinant protein or cell-surface panning using a monovalent scFv phage display library is described (Schier et al. J. Mol. Biol. 255:28, 1996; Chames et al., pp 149-166 in R. Kontermann & S. Duebel, editors (2001) Antibody Engineering (Springer Laboratory Manual), Springer Verlag). Using the high diversity, naïve phagemid library along with conventional panning techniques the isolation of a number of antibodies to recombinant TR was achieved, however these failed to recognize native cell-surface TR by FACS analysis. Cell-surface panning was then attempted since it is a constructive alternative to conventional panning for the isolation of antibodies to native protein. Using a single TR over-expressing cell-line, (PC3-TR with 0.65×10⁶ TR/cell) and a helper-phage-rescued monovalent phagemid library, only non-TR binding clones were identified even though selections were always preceded by an adsorption step on TR-negative parental PC3 cells. These non-specific clones bound both the parental cell-line and the TR recombinant cell-line indicating that the antibodies were specific for some component of the complex matrix of the cell membrane but were not specific for TR.

In addition, a high number of insert-less phage were observed in the phage population recovered during the third round of panning with this single PC3-TR cell-line. Such difficulties with cell-surface panning have been previously described (Hegmans et al., J. Immun. Methods 262:191, 2002). These failures required an alternative approach to isolate potent anti-TR antibodies by phage display technology.

To circumvent these obstacles, the original monovalent phagemid library was rescued with hyperphage to render the library multivalent. Multivalency is reported to increase the selection efficiency of phage display technology due to a dramatic increase in display frequency and avidity effects (Rondot et al., Nature Biotech. 19:75, 2001; O'Connell et al., J. Mol. Biol. 32:149, 2002; Chingwei et al., J. Immun. Methods 284:119, 2003). Hyperphage also reduced the occurrence of insert-less phage. After three rounds of panning with hyperphage rescue at each round, 100% of clones were shown to have scFv insert at the genetic level. In addition, the titers of eluted phage increased by about one log with each round of panning as seen in Table II indicating a strong selection pressure.

To decrease the enrichment of non-specific phage, a selection strategy using two TR-positive cell-lines with different background matrices was used. For this approach a recombinant cell line (CHO-TR) was created by transforming CHO-K1 cells with an expression vector for TR. The resulting cell line had expression levels of 1.8×10⁶ TR receptors per cell. The second cell line used was a human PC3-TR cell line (0.65×10⁶ TR receptors per cell) made by transforming human PC3 cells with the TR expression vector. The background matrices for the TR-antigen are different in these cell lines because they are derived from different species, CHO-TR from hamster and PC3-TR from human. When these cell lines are used in alternate rounds of panning (CHO-TR, PC3-TR, CHO-TR, etc.), the alternation of the cell lines serves to deplete the library of any matrix-binding clones (i.e. any non-TR binding clones). In addition, pre-adsorption of the library on parental cells at each round of panning (CHO, PC3, CHO) depletes the library of non-specific antibodies.

A multivalent reverse-capture ICELISA was developed for the primary screen to identify all TR-binding clones in the round three polyclonal scFv pool. In this screening method, an anti-E-tag IgG ELISA plate captures the E-tagged scFv from crude bacterial culture, effectively creating a surface population of the bivalent scFv in the assay. The 6×his-tagged recombinant TR protein is bound and TR binding is detected with an anti-6×his-HRP antibody. Even if the scFv binds the TR protein weakly, TR will be captured due to the avidity effect from the use of the bivalent scFv.

The fact that a single TR-binding clone was identified could be a reflection of a limited number of clones in the library that recognize native TR on cells. It may also result from the high stringency panning method employed for this study. A higher diversity of TR-binding clones may result if fewer wash steps and fewer library pre-adsorption steps are employed. Literature reports recommend high stringency and many washes (Mutuberria et al., J. Imm. Methods 231:65, 1999; Tur et al., Biotechniques 30:404, 2001) to eliminate non-specific phage as described here while others report optimization with significantly fewer washes (Pereira et al., J. Immun. Methods 203:11, 1997; Hegmans et al., J. Immun. Methods 262:191, 2002).

No non-specific clones recognizing the parental cell-line were identified. This underscores the efficiency of the counter selection and the alternation of the two TR-positive cell-lines in completely removing these species.

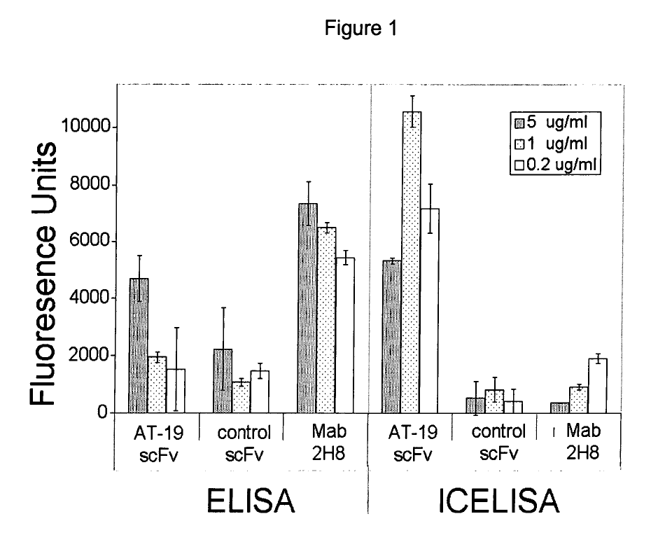

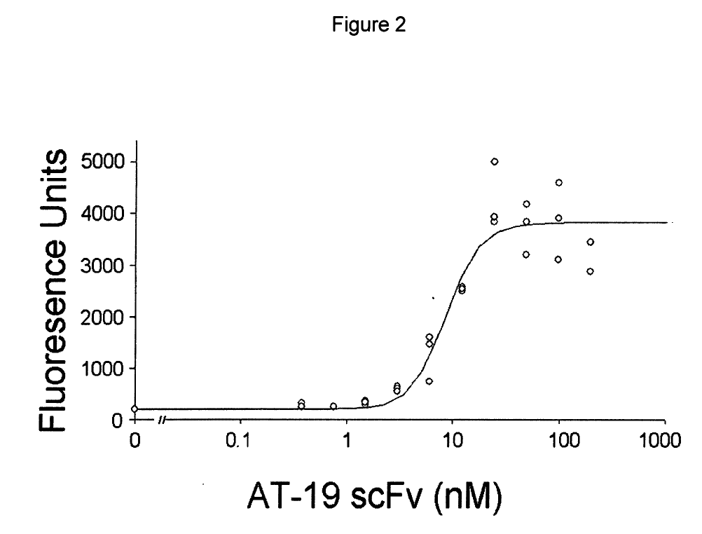

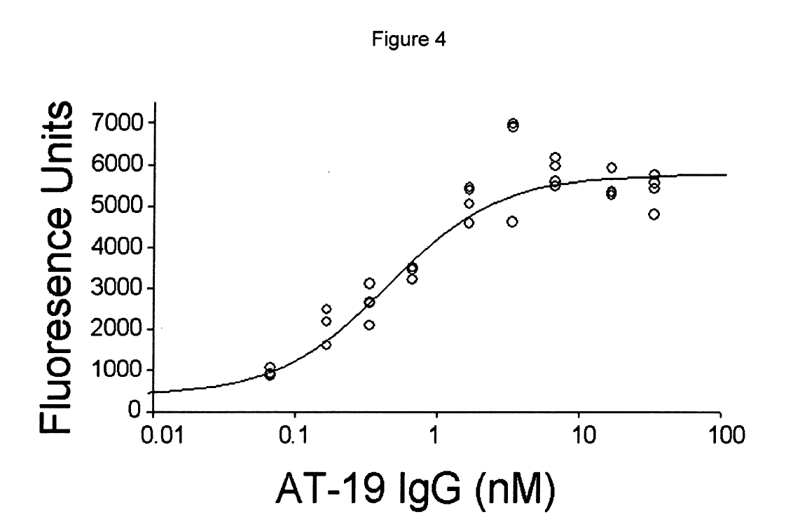

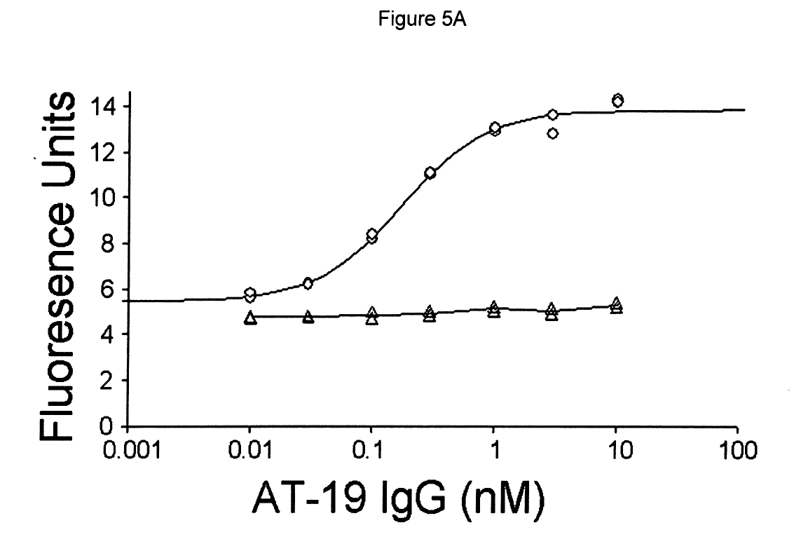

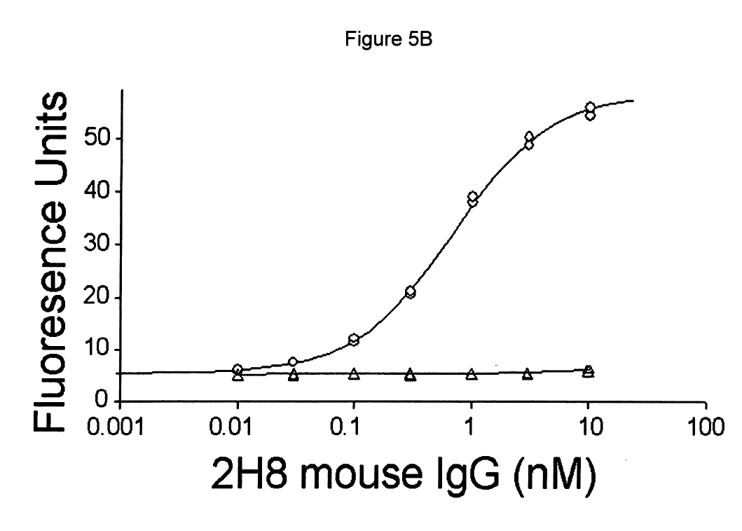

FIG. 6 shows the amino acid sequence of the single chain (scFv) of the anti-TR antibody, AT-19 (SEQ ID NO:1) and delineates the V_{H}-V_{L} linker, the V_{H} and V_{L} domains, and the CDR regions. Characterization of the pure single chain form of the anti-TR antibody, AT-19, reveals that bivalency either via immunocomplex or conversion to the IgG format is required for binding of the antibody to the native receptor. The purified monovalent single-chain antibody failed to bind to the native receptor in a cell-based ELISA significantly better than the control scFv, even when incubated at concentrations between 8 and 200 nM scFv. When rendered bivalent through an immunocomplex with anti-Etag IgG, AT-19 scFv is shown to bind to TR-expressing cells (FIG. 1, panel B). The confirmation that the AT-19 antibody binds only as a dimer is further illustrated upon conversion to the IgG format. An even greater avidity effect is observed for the IgG than for the scFv immunocomplex (comparison of FIG. 2 to FIGS. 4 and 5A). This is likely due to the ease of determination of the exact concentration of AT-19 IgG in the live cell ELISA and FACS assay as compared to the poorly determined concentration of the (scFv)₂/IgG ternary complex in the live cell ICELISA. AT-19 IgG exhibited an EC₅₀ of 0.44 nM by cell-ELISA and 0.17 nM by FACS (FIGS. 4 and 5A). These values are the true measure of the avidity of AT-19 and are noteworthy since the monovalent antibody did not bind the native TR even at high concentrations. For comparison the mouse anti-TR IgG 2H8 was analyzed similarly and found to have an affinity of 0.67 nM, which agrees with the Biacore-determined affinity of 1 nM (Uchida et al., BBRC 266:593, 1999).

AT-19 does not bind native TR as the scFv monomer, making it impossible to select AT-19 from a purely monovalent phage-display library. Rescue with hyperphage yields phage particles bearing multiple pill-scFv fusions per phage and multivalent phage were required to isolate AT-19. The phage particle of the multivalent phage fulfills a similar role as the anti-Etag IgG in the immunocomplex and as the constant region of the IgG. All three approaches serve to present at least two copies of the complex of V_{H} and V_{L} to the antigen. For the multivalent phage and immunocomplex, the two complexes are presented as the scFv format and for the IgG, the complexes of VH and VL are on the two arms of the bivalent antibody. Multivalent phage display can be viewed as a prerequisite for the isolation of antibodies that dissociate rapidly. In the absence of the avidity gain conferred by multivalency, phage antibodies such as AT-19 are lost in the washing steps designed to eliminate non-binding phage.

AT-19 IgG is shown to selectively internalize into TR-expressing cells (see Example 11). The AT-19 antibody exhibits the ability to internalize within cells that over-express the TR receptor. The AT-19 antibody can, therefore, be used to selectively target and destroy TR expressing cells by conjugation of the antibody to a cytotoxic drug.

Antibody Fragments

Antibody fragments which contain specific binding sites for TR may also be generated. There are often advantages to using antibody fragments, rather than whole antibodies, since the smaller size of the fragments can lead to more rapid clearance, and may also provide improved access to solid tumors.

Such fragments include, but are not limited to the F(ab′)₂ fragments which can be produced by pepsin digestion of the IgG antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science 256:1270-1281, 1989). Fab, Fv and ScFv antibody fragments can all be expressed in and secreted from E. coli, or a variety of eukaryote cell expression systems, allowing for the production of large amounts of these fragments. Alternatively, Fab′-SH fragments can be directly recovered from E. coli and chemically coupled to form F(ab′)₂ fragments (Carter et al., Bio/Technology 10:163, 1992). Other techniques for the production of antibody fragments are known to those skilled in the art. Single chain Fv fragments (scFv), diabodies, minibodies and other engineered antibody fragments are also envisioned (see U.S. Pat. Nos. 55,761,894 and 5,587,458; Hudson et al. Nature Medicine 9:129, 2003). Fv and sFv fragments are examples of species with intact combining sites that are devoid of constant regions; thus, they are likely to show reduced nonspecific binding during in vivo use, and are particularly preferred for use as imaging agents (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). The antibody fragment may also be a “linear antibody”, as described in U.S. Pat. No. 5,641,870, for example. Such linear antibody fragments may be monospecific or bispecific.

Variants of the antibodies or antibody fragments described herein are also contemplated, and can be made using any of the techniques and guidelines for conservative and non-conservative mutations, e.g. U.S. Pat. No. 5,364,934. Variations include substitution, deletion or insertion of one or more codons encoding the antibody, resulting in a change in the amino acid sequence as compared with the native antibody sequence. The utility of such variations contemplated would include those leading to (1) a reduction in susceptibility to proteolysis or inactivation by oxidation, (2) an alteration in binding affinity for forming protein complexes or binding affinities to antigens, (3) an alteration in in vivo clearance or biodistribution, (4) changes in the antibody isotype or allotype, (5) changes in the functional properties of the antibody, for example Fc receptor binding, (6) an alteration in epitope sequences to decrease or increase immunogenicity, (7) alteration in the linker length to improve scFv dimer (diabody) stability, and (8) other changes in physiocochemical or functional properties of such analogs. Guidance in determining which amino acid residue may be inserted, substituted or deleted without adversely affecting the desired activity may be found by minimizing the number of amino acid sequence changes made in regions of high homology between the TR antibodies and that of homologous proteins. The variation allowed may be determined by systematically making insertions, deletions or substitutions of amino acids in the sequence and testing the resulting variants for activity exhibited by the native sequence.

A particularly preferred type of substitutional variant involves substituting one more hypervariable region residues of a parent antibody (e.g. human antibody). Generally, the resulting variant(s) selected for further development will have improved biological properties relative to the parent antibody from which they are generated. A convenient way for generating such substitutional variants involves affinity maturation using phage display (Schier R., J. Mol. Biol., 263:551-67, 1996). The variants are then screened for their biological activity (e.g. binding affinity) as described herein (see Example 5). In order to identify hypervariable region residues which would be good candidates for modification, alanine scanning mutagenesis can be performed to identify hypervariable region residues contributing significantly to antigen binding. Antibodies with superior properties in one or more relevant assays can undergo further development.

Variants also include antibodies that differ in amino acid sequence due to the insertion or deletion of amino acid residues by mutagenesis. For example, mutagenesis may be performed according to recombinant DNA techniques well known in the art to insert or delete amino acids in the N-terminal or C-terminal portions of the V_{H} or V_{L} domains of the Ig light and heavy chains to create variants that retain substantially similar functional activity. Particularly preferred variants include single chain antibodies with insertions or deletions of amino acid residues in the V_{H}-V_{L} linker between the V_{H} and V_{L} domains. Linker sequences, from 0 to 20 amino acids may be used, wherein the antibody retains substantially similar functional activity. Modifications of the existing V_{H}-V_{L} linker may be aimed at increasing the stability of the dimer form of the single chain antibody.

Several techniques are known in the art for screening gene products of combinatorial libraries made by point mutations or truncation, and for screening cDNA libraries for gene products having a selected property. The most widely used techniques, which are amenable to high throughput analysis for screening large gene libraries typically include cloning the gene library into replicable expression vectors, transforming appropriate cells with the resulting library of vectors and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify the desired variants.

Use of Antibodies Recognizing Epitopes of TR

The antibodies, antigen-binding antibody fragments, and variants thereof of the invention may be particularly useful in diagnostic assays, imaging methodologies, and therapeutic methods for the management of cancers in which TR is over-expressed, including, but not limited to, cancer of the prostate. Antibodies which bind TR may modulate TGFβ and non-TGFβ mediated signaling pathways controlled by TR with or without its co-receptor Cripto. (Changs and Harms, Genes and Development 17:2624, 2003).

The invention provides various immunological assays useful for the detection of TR polypeptides and for the diagnosis of cancers in which TR is over-expressed including, but not limited to, prostate cancer. Such assays generally comprise one or more TR antibodies capable of recognizing and binding a TR polypeptide. The most preferred antibodies will selectively bind to TR and will not bind (or bind weakly) to non-TR polypeptides. The assays include various immunological assay formats well known in the art, including but not limited to various types of radioimmunoassays, enzyme-linked immunoabsorbent assays, and the like. In addition, immunological imaging methods capable of detecting prostate cancer are also provided by the invention, including but not limited to radioscintigraphic imaging methods using labeled TR antibodies. Such assays may be clinically useful in the detection, monitoring and prognosis of cancers, including but not limited to proatate cancer. The above-described antibodies may be employed to isolate or to identify clones expressing the polypeptide or to purify the polypeptide of the present invention by attachment of the antibody to a solid support for isolation and/or purification by affinity chromatography.

Additionally, TR antibodies may be used to isolate TR positive cells using cell sorting and purification techniques. In particular, TR antibodies may be used to isolate prostate cancer cells from xenograft tumor tissue, from cells in culture, etc. using antibody-based cell sorting or affinity purification techniques. Other uses of the TR antibodies of the invention include generating anti-idiotypic antibodies that mimic the TR polypeptide.

TR antibodies can be used for detecting the presence of prostate cancer or tumor metastasis. The presence of such TR-containing cells within various biological samples, including serum, prostate and other tissue biopsy specimens, may be detected with TR antibodies. In addition, TR antibodies may be used in various imaging methodologies such as immunoscintigraphy with a ^{99m}Tc (or other isotope) conjugated antibody. For example, an imaging protocol similar to the one recently described using an ¹¹¹In conjugated anti-PSMA antibody may be used to detect recurrent and metastatic prostate carcinomas (Sodee et al., Clin. Nuc. Med. 21: 759-766, 1997). Another method of detection that can be used is positron emitting tomography (see Herzog et al., J. Nucl. Med. 34:2222-2226, 1993).

The TR antibodies of the invention may be labeled with a detectable marker or conjugated to a second molecule, such as a cytotoxic agent, and used for targeting the second molecule to a TR positive cell (Vitetta, E. S. et al., Immunotoxin Therapy, in DeVita, Jr, V. T. et al., eds, Cancer: Principles and Practice of Oncology, 4^{th} ed., J.B. Lippincott Co., Philadelphia, 2624-2636, 1993). Examples of cytotoxic agents include, but are not limited to ricin, doxorubicin, daunorubicin, paclitaxol (TAXOL™), ethidium bromide, mitomycin, etoposide, tenoposide, vincristine, vinblastine, colchicine, dihydroxy anthracin dione, actinomycin D, diptheria toxin, epothilones, monomethyl auristatin E (MMAE), Pseudomonas exotoxin (PE) A, PE40, abrin, and glucocorticoid and other chemotherapeutic agents, as well as radioisotopes. Cytotoxic or antiproliferative targeted fusion proteins may be created by genetic or chemical fusion of the antibody to an appropriate cytokine, chemokine, interferon, or growth factor that has the desired anti-tumor biological activity (Asgeirsdottir et al., Biochem. Pharmacol. 15:1729-1739, 2003). Suitable detectable markers include, but are not limited to, a radioisotope, a fluorescent compound, a bioluminescent compound, chemiluminescent compound, a metal chelator or an enzyme. Suitable radioisotopes for immunotherapy or for use as a detectable marker include the following: Antimony-124, Antimony-125, Arsenic-74, Barium-103, Barium-140, Beryllium-7, Bismuth-j206, Bismuth-207, Cadmium-109, Cadmium-115m, Calcium-45, Cerium-139, Cerium-141, Cerium-144, Cesium-137, Chromium-51, Cobalt-56, Cobalt-57, Cobalt-58, Cobalt-60, Cobalt-64, Erbium-169, Europium-152, Gadolinium-153, Gold-195, Gold-199, Hafnium-175, Hafnium-181, Indium-111, Iodine-123, Iodine-131, Iridium-192, Iron-55, Iron-59, Krypton-85, Lead-210, Lutetium-177, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-2226, Rhenium-186, Rubidium-86, Ruthenium-103, Ruthenium-106, Scandium-44, Scandium-46, Selenium-75, Silver-110m, Silver-11, Sodium-22, Strontium-85, Strontium-89, Strontium-90, Sulfur-35, Tantalum-182, Technetium-99m, Tellurium-125, Tellurium-132, Thallium-170, Thallium-204, Thorium-228, Thorium-232, Tin-113, Titanium-44, Tungsten-185, Vanadium-48, Vanadium-49, Ytterbium-169, Yttrium-88, Yttrium-90, Zinc-65, and Zirconium-95.

Radiolabeling of antibodies can be accomplished using a chelating agent which is covalently attached to the antibody, with the radionuclide inserted into the chelating agent. Preferred chelating agents are set forth in Srivagtava et al. Nucl. Med. Bio. 18:589-603, 1991 and McMurry et al., J. Med. Chem. 41:3546-3549, 1998. or derived from the so-called NOTA chelate published in H. Chong, K. et al., J. Med. Chem. 45:3458-3464, 2002, all of which are incorporated herein in full by reference.

Particularly preferred for use as a detectable marker for immunoscintigraphy are the radioisotopes ¹¹¹In or ^{99m}Tc. Preferred detectable markers for positron emitting tomography are ⁴³Sc, ⁴⁴Sc, ⁵²Fe, ⁵⁵Co, ⁶⁸Ga, ⁵⁴Cu, ⁸⁶Y and ^{94m}Tc. For immunotherapy, the beta-emitting radioisotopes ⁴⁶Sc, ⁴⁷Sc, ⁴⁸Sc, ⁷²Ga, ⁷³Ga, ⁹⁰Y, ⁶⁷Cu, ¹⁰⁹Pd, ¹¹¹Ag, ¹⁴⁹Pm, ¹⁵³Sm, ¹⁶⁶Ho, ¹⁷⁷Lu, ¹⁸⁶Re, and ¹⁸⁸Re and the alpha-emitting isotopes ²¹¹At, ²¹¹Bi, ²¹²Bi, ²¹³Bi and ²¹⁴Bi, can be used. Preferred are ⁹⁰Y, ¹⁷⁷Lu, ⁷²Ga, ¹⁵³Sm, 67Cu and ²¹²Bi, and particularly preferred are ⁹⁰Y and ¹⁷⁷Lu.

Immunotherapy for Prostate and Other Cancers

The invention provides various immunotherapeutic methods for treating prostate and other cancers, including antibody therapy, in vivo vaccines, and ex vivo immunotherapy approaches. In one approach, the invention provides TR antibodies which may be used systemically to treat prostate cancer. For example, unconjugated TR antibodies may be introduced into a patient such that the antibody binds to TR on, in or associated with prostate cancer cells and mediates the destruction of the cells, and the tumor, by mechanisms which may include complement-mediated cytolysis, antibody-dependent cellular cytotoxicity, altering the physiologic function of TR, and/or the inhibition of ligand binding or signal transduction pathways. TR antibodies conjugated to toxic agents such as ricin or radioisotopes, as described above, may also be used therapeutically to deliver the toxic agent directly to TR-bearing prostate tumor cells and thereby destroy the tumor cells.

Prostate cancer immunotherapy using TR antibodies may follow the teachings generated from various approaches which have been successfully employed with respect to other types of cancer, including but not limited to colon cancer (Arlen et al., Crit. Rev. Immunol. 18: 133-138, 1998), multiple myeloma (Ozaki et al., Blood 90: 3179-3186, 1997; Tsunenari et al., Blood 90: 2437-2444, 1997), gastric cancer (Kasprzyk et al, Cancer Res. 52: 2771-2776, 1992), B-cell lymphoma (Funakoshi et al., Immunther. Emphasis Tumor Immunol. 19: 93-101, 1996), leukemia (Zhong et al., Leuk. Res. 20: 581-589, 1996), colorectal cancer (Moun et al., Cancer Res. 54: 6160-6166, 1994; Velders et al., Cancer Res. 55:4398-4403, 1995), and breast cancer (Shepard et al., J. Clin. Immunol. 11: 117-127, 1991).

The invention further provides vaccines formulated to contain a TR polypeptide or fragment thereof. The use of a tumor antigen in a vaccine for generating humoral and cell-mediated immunity for use in anti-cancer therapy is well known in the art and has been employed in prostate cancer using human PSMA and rodent PAP immunogens (Hodge et al., Int. J. Cancer 63: 231-237, 1995; Fong et al., J. Immunol. 159: 3113-3117, 1997). Such methods can be readily practiced by employing an TR polypeptide, or fragment thereof, or an TR-encoding nucleic acid molecule and recombinant vectors capable of expressing and appropriately presenting the TR immunogen.

For example, viral gene delivery systems may be used to deliver a TR-encoding nucleic acid molecule. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbis virus (Restifo, in Curr. Opin, Immunol. 8: 658-663, 1996). Non-viral delivery systems may also be employed by using naked DNA encoding a TR polypeptide or fragment thereof introduced into the patient (i.e., intramuscularly) to induce an anti-tumor response. In one embodiment, the full-length human TR cDNA may be employed. In another embodiment, human TR cDNA fragments may be employed. In another embodiment, TR nucleic acid molecules encoding specific T lymphocyte (CTL) epitopes may be employed. CTL epitopes can be determined using specific algorithims (e.g., Epimer, Brown University) to identify peptides within a TR polypeptide which are capable of optimally binding to specified HLA alleles.

Various ex vivo strategies may also be employed. One approach involves the use of dendritic cells to present a TR polypeptide as antigen to a patient's immune system. Dendritic cells express MHC class I and II, B7 costimulator, and IL-12, and are thus highly specialized antigen presenting cells. In prostate cancer, autologous dendritic cells pulsed with peptides of the prostate-specific membrane antigen (PSMA) are being used in a Phase I clinical trial to stimulate prostate cancer patients' immune systems (Tjoa et al., Prostate 28: 65-69, 1996; Murphy et al., Prostate 29: 371-380, 1996). Dendritic cells can be used to present TR polypeptides to T cells in the context of MHC class 1 and 11 molecules. In one embodiment, autologous dendritic cells are pulsed with TR polypeptides capable of binding to MHC molecules. In another embodiment, dendritic cells are pulsed with the complete TR polypeptide. Yet another embodiment involves engineering the overexpression of the TR gene in dendritic cells using various implementing vectors known in the art, such as adenovirus (Arthur et al., Cancer Gene Ther. 4: 17-25, 1997), retrovirus (Henderson et al., Cancer Res. 56: 3763-3770, 1996), lentivirus, adeno-associated virus, DNA transfection (Ribas et al., Cancer Res. 57: 2865-2869, 1997), and tumor-derived RNA transfection (Ashley et al., J. Exp. Med. 186: 1177-1182, 1997).

Anti-idiotypic anti-TR antibodies can also be used in anti-cancer therapy as a vaccine for inducing an immune response to cells expressing an TR polypeptide. Specifically, the generation of anti-idiotypic antibodies is well known in the art and can be readily adapted to generate anti-idiotypic anti-TR antibodies that mimic an epitope on a TR polypeptide (see, for example, Wagner et al., Hybridoma 16: 33-40, 1997: Foon et al., J. Clin. Invest. 96: 334-342, 1995; Herlyn et al., Cancer Immunol Immunother 43: 65-76, 1996). Such an anti-idiotypic antibody can be used in anti-idiotypic therapy as presently practiced with other anti-idiotypic antibodies directed against tumor antigens.

Genetic immunization methods may be employed to generate prophylactic or therapeutic humoral and cellular immune responses directed against cancer cells expressing TR. Using the TR-encoding DNA molecules described herein, constructs comprising DNA encoding an TR polypeptide/immunogen and appropriate regulatory sequences may be injected directly into muscle or skin of an individual, such that the cells of the muscle or skin take up the construct and express the encoded TR polypeptide/immunogen. The TR polypeptide/immunogen may be expressed as a cell surface polypeptide or be secreted. Expression of the TR polypeptide/immunogen results in the generation of prophylactic or therapeutic humoral and cellular immunity against prostate cancer. Various prophylactic and therapeutic genetic immunization techniques known in the art may be used.

Pharmaceutical Compositions and Administration

The present invention also relates to pharmaceutical compositions which may comprise TR polynucleotides, TR polypeptides, antibodies, agonists, antagonists, or inhibitors, alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxilliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxilliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, ie. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

In another embodiment, the kits may contain DNA sequences encoding the antibodies of the invention. Preferably the DNA sequences encoding these antibodies are provided in a plasmid suitable for transfection into and expression by a host cell. The plasmid may contain a promoter (often an inducible promoter) to regulate expression of the DNA in the host cell. The plasmid may also contain appropriate restriction sites to facilitate the insertion of other DNA sequences into the plasmid to produce various antibodies. The plasmids may also contain numerous other elements to facilitate cloning and expression of the encoded proteins. Such elements are well known to those of skill in the art and include, for example, selectable markers, initiation codons, termination codons, and the like.

Manufacture and Storage.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with may acids, including by not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition. For administration of TR, such labeling would include amount, frequency and method of administration.

Therapeutically Effective Dose.

Pharmaceutical compositions suitable for use in the present invention include compositions wherein the active ingredients are contained in an effective amount to achieve the intended purpose, i.e. treatment of a particular disease state characterized by TR expression. The determination of an effective dose is well within the capability of those skilled in the art.

For any compound, the therapeutically effective dose can be estimated initially either in cell culture assays, e.g., neoplastic cells, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of protein or its antibodies, antagonists, or inhibitors that ameliorate the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies is used in formulating a range of dosage for human use. The dosage of such compounds lies preferably within a range of circulating concentrations what include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, eg, tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature. See U.S. Pat. No. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc. Preferred specific activities for for a radiolabeled antibody may range from 0.1 to 10 mCi/mg of protein (Riva et al., Clin. Cancer Res. 5:3275s-3280s, 1999; Wong et al., Clin. Cancer Res. 6:3855-3863, 2000; Wagner et al., J. Nuclear Med. 43:267-272, 2002).

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

### EXAMPLES

1. Recombinant TR Protein Production

The extracellular domain of TR comprising amino acids 1-302 was expressed as a secretory protein in the baculovirus expression system. Briefly, the cDNA encoding the extracellular domain was amplified with primers encoding an N-terminal optimized Kozak sequence and a C-terminal 6×-His tag which is preceded by two glycine residues and followed by a stop codon. The PCR product was digested with the restriction enzymes BamHI and KpnI and cloned downstream of the polyhedrin promoter into the baculovirus transfer vector pBBS250. Recombinant baculovirus was produced by cotransfection with BaculoGold™ DNA (BD Biosciences, San Diego, Calif.) and subsequent plaque isolation. High Five™ cells were infected at an MOI of 3 and the cell culture supernatant was harvested after 72 hr. The protein was purified in a two-step procedure by Ni2+-chelating and size exclusion chromatography. The protein exhibits correct processing of the signal sequence after amino acid Ala₄₀ and is glycosylated.

2. Generation of Cell Lines

The human prostate carcinoma cell-line PC3 and the Chinese Hamster Ovary cell-line CHO-K1 were purchased from American Type Culture Collection (Manassas, Va.). PC3 cells were grown in RPMI-1640 medium, supplemented with 10% fetal bovine serum (FBS), L-glutamine and Penicillin/Streptomycin, at 37° C. in a humidified atmosphere of 5% CO₂. CHO-K1 cells were maintained in ATCC complete growth medium (Ham's F12K medium with 2 mM L-glutamine adjusted to contain 1.5 g/L sodium bicarbonate, 90%; fetal bovine serum, 10%). Stable transfection of PC3 or CHO-K1 cells with the expression vector pcDNA3.1-TR generated the PC3-TR and CHO-TR cell lines, respectively. These cells were maintained in growth medium, supplemented with 400 μg/ml G418. Cells were characterized by flow cytometry and internalization assay. Based on Scatchard analysis, PC3-TR cells express 0.65 million TR molecules per cell and CHO-TR cells express 1.8 million TR molecules per cell.

3. scFv Library Generation

The scFv library generation was based upon published reports (Marks pp 53-88 in C. A. K. Borrebaeck, editor (1995) Antibody Engineering, Oxford University Press; Sheets et al., Proc. Natl. Acad. Sci. 95:6157, 1998). The variable genes (V_{H}, V_{K} and V_{λ}) were PCR cloned from pooled mRNA from human bone marrow, lymph node and spleen using a set of family specific primers. The resultant PCITE-V_{H} (3.9×10⁹ members), PZ604-V_{K} (1.6×10⁷) and PZ604-V_{λ} (3.2×10⁷) libraries represent a permanent and high diversity source of V genes. The V_{H} genes were PCR amplified from PCITE-V_{H} library and the V_{λ} and V_{λ} genes were PCR amplified from the pZ604-V_{K} and PZ604-V_{λ} library together with the reverse J_{H} and linker sequence at the 5′ end. The gel purified V_{H}, V_{K} and V_{λ} genes containing PCR products were then spliced together by overlap extension PCR to make the scFv gene repertoire. The scFv gene repertoire was cloned into a phagemid vector pZ603, and the ligation product was electroporated into competent TG1 E. coli cells to generate the scFv phage display library, HuPhabL3, with 5.2×10⁹ individual transformants. Pz603 is based upon the commercially available pCANTAB5E (Amersham GE Healthcare, Piscataway, N.J.) with an NcoI site introduced 5′ to the VH region. A high diversity was indicated by the library size of 5.2×10⁹ along with a good display level of >33%. The library was rescued with hyperphage (Research Diagnostics, Inc., Concord, Mass.) at an MOI of 20, phage was precipitated by polyethylene glycol/NaCl precipitation, and purified by standard methods. The hyperphage-rescued library was used as the input for the first round of panning.

4. Differential Cell Panning

Three rounds of panning with the multivalent, hyperphage rescued library were designed to alternate the cell-line used to present the TR antigen and to pre-absorb non-specific phage on TR negative cells. Round 1: CHOK1 parental cells (1×10⁸) were blocked with 10 mL 2% BSA, 10% FBS in PBS (blocking buffer) for 1 hr at 4° C. The cells were divided into 3 tubes and pelleted at 1000 rpm for 5 min. The multivalent scFv phage display library, diluted in 5 mL blocking buffer, was incubated sequentially for 30 min with each tube of parental cells at 4° C. while rocking. After the 3^{rd} depletion step on the CHOK1 parental cells the library (supernatant) was added to CHO-TR cells (1×10⁷) for selection on the positive cell-line for 2 hr at 4° C. with rocking. The cells were then washed 10 times with 10 mL blocking buffer changing tubes with each wash. Phage which remained bound were eluted by lysing the cells with 0.5 mL of 100 mM triethylamine (TEA) for 10 min followed by neutralization with 1 mL of 1 M tris buffer, pH 7.5. Round 2: The same procedure as described for round 1 except 15 washes were performed after selection on the positive cell-line. PC3 parental cells and PC3-TR positive cells were used instead of CHOK1 and CHO-TR cells. Round 3: The same procedure as described for round 1 except 20 washes were performed after selection on the positive (CHO-TR) cell-line. Phage amplification and purification were performed by standard methods (Rider et al. pg/62, in B. K. Kay, J. Winter and J. McCafferty (Editors) (1996) Phage Display of Peptides and Proteins, Academic Press). Briefly, eluted phage from each round were amplified by infection of 15 mL log-phase TG1 with 0.75 mL of eluate. TG1 were grown at 37° C. to an OD₆₀₀ of 0.7 at 37° C. After addition of the eluate, TG1 were incubated at 37° C. for 30 min. 0.1, 1 and 10 μL of the culture were plated onto 10 cm agar plates for determination of phage titer. Phage titer indicates the number of phage particles in the eluate or phage stock. The bacterial cells were then pelleted by centrifugation at 3000 rpm, the supernatant was removed and the cell pellet spread onto two 15 cm agar plates with 2XTY, carbenicillin (100 μg/mL), 1% glucose. The plates were incubated at 37° C. overnight. Glycerol stocks of the bacteria were prepared by scraping the colonies off the plates using a total of 4 mL of 2XTY, carbenicillin (100 μg/mL), 1% glucose, 17% glycerol and were stored at ˜0° C. For rounds 1 and 2, 13 mL of 2XTY, carbenicillin (100 μg/mL), 1% glucose was inoculated with an aliquot of the glycerol stock bringing the starting OD₆₀₀ to approximately 0.05. The culture was grown to log phase at 37° C., infected with hyperphage (Research Diagnostics, Inc., Concord, Mass.) at a MOI of 20 and then incubated for two 45 min periods at 37° C., first stationary and then with agitation. The culture was centrifuged at 3000 rpm to pellet the bacteria, the supernatant removed and the bacterial pellet resuspended in 50 mL 2XTY, carbenicillin (100 μg/mL), kanamycin (50 μg/mL). The culture was grown overnight at 28° C. Phage were purified from the overnight culture by standard methods.

5. Primary Screening by Reverse Capture ICELISA and Differential Cell-Based ELISA

Single colonies from the 3^{rd} round of panning were picked into 96-well plates, and plates expressing soluble scFv in single-clone format were prepared for screening. ScFv were expressed by growth of clones to log-phase under selection in carbenicillin followed by induction with 1 mM IPTG. After induction plates were grown overnight at 28° C. using a GeneMachines HiGro high capacity incubator (Genomic Solutions, Ann Arbor, Mich.) in an O₂ atmosphere with shaking at 500 rpm. The next day, plates were spun at 3000 rpm to pellet bacteria and the supernatant was used for ELISA screening.

Immunocomplex ELISA (ICELISA): The ICELISA is adapted from the method described by Amersham GE Healthcare in the RPAS phage display system. Screening was performed on 6×his-tagged recombinant TR expressed in insect cells as described above. Anti-Etag-antibody (Amersham GE Healthcare, Piscataway, N.J.) was coated onto the ELISA plate overnight at 4° C. at a concentration of 1 μg/mL. The ELISA plate was then blocked with 5% Milk-PBS for an hour and then washed once. 100 μL of bacterial cell culture supernatant was transferred from the expression plate to the ELISA plate to capture the antibody. ELISA plates were incubated for 1 hr at room temperature. Plates were washed three times in 0.05% TPBS. Recombinant TR (100 μL, 2 mg/mL) was added for 1 hr at room temperature followed by three washes. TR was probed with anti-6×his-HRP. After three final washes the plate was developed with Amplex Red reagent (Molecular Probes Invitrogen, Eugene, Oreg.) and read at Ex₅₃₀/Em₅₉₀ on a Gemini EM fluorometer (Molecular Devices, Sunnyvale, Calif.).

Differential cell ELISA: Screening was performed on CHO-TR and on CHOK1 cells grown adherently to confluence on collagen I 96-well cell culture plates (BD Biocoat, BD Biosciences, San Diego, Calif.). 100 μL of bacterial cell culture supernatant from the expression plate was then transferred to the cell ELISA plate. ELISA plates were incubated for 1 hr at 4° C. The plates were washed three times with cold PBS containing calcium and magnesium (GIBCO Invitrogen, Carlsbad, Calif.). anti-Etag-HRP (Amersham GE Healthcare, Piscataway, N.J.) diluted in blocking buffer to a final dilution of 1:5000 was added for 1 hr at 4° C. followed by three washes in cold PBS. The ELISA plate was developed with Amplex Red HRP substrate and read in the fluorometric plate reader (Ex₅₃₀/Em₅₉₀).

6. ScFv Expression and Purification

HB2151 E. coli cells transfected with an scFv gene of interest were grown in 1 L of media to log phase (OD₆₀₀=0.7), induced with 0.75 mM IPTG and grown overnight at 28° C. The scFv was extracted from the periplasm using B-PER bacterial protein extraction reagent (Pierce Biotechnology, Rockford, Ill.). The scFv was purified from the sterile-filtered supernatant using the RPAS purification module (Amerhsham GE Healthcare, Piscataway, N.J.) which relies on E-tag affinity chromatography.

7. Format Switching: from scFv to IgG

The expression vector of pIE_SRγ1fa (Medarex, Inc., Milpitas, Calif.) contains cDNA's encoding the CH and CL regions of human IgG1 (fa haplotype) and kappa chains, respectively. The cDNA's encoding the variable regions (VL (SEQ ID NO:6) and VH (SEQ ID NO:7)) of anti-TR antibody AT-19 (see FIG. 7) were fused to an IgG signal sequence first by a PCR method. The PCR method generated the VL and VH cDNA's that were then cloned into pIE_SRγ1fa such that the VL and VH were in frame with the CL and CH region in the respective pIE derivatives. Primers used for the in-frame cloning were:

| | |
|---|---|
| F-ss-VL: | |
| caggaagcttgccaccatggaaaccccagcgcagct | (SEQ ID NO:19) |
| tctcttcctcctgctactctggctc | |
| R-ss-VL: | |
| agtcagtataacgtgactagttccggtggtatctgg | (SEQ ID NO:20) |
| gagccagagtagcaggaggaagagaag | |
| F-VL: | |
| actagtcacgttatactgactcaaccgccctc | (SEQ ID NO:21) |
| R-VL: | |
| cgtgcgtacgacctaggacggtcagcttggtccc | (SEQ ID NO:22) |
| F-ss-VH: | |
| ataagaatgcggccgccaccatggagtttgtgctga | (SEQ ID NO:23) |
| gctgggttttccttgttgctata | |
| R-ss-VH: | |
| ctgcaccagctgcacctggatatcacactggacacc | (SEQ ID NO:24) |
| ttttaatatagcaacaaggaaaacccagctcagc | |
| F-VH: | |
| gatatccaggtgcagctggtgcagtctgg | (SEQ ID NO:25) |
| R-VH: | |
| ctgccgctagccgagacggtgaccagggttc | (SEQ ID NO:26) |

The final construct was named as pIE/TR_Ab. pIE/TR_Ab and was transfected into HEK 293 EBNA cells in suspension. Conditioned medium was harvested 3 days post transfection for down-stream purification.

8. AT-19 IgG Purification

Conditioned media from HEK 293 EBNA cells was recovered after 3 days of transient transfection by centrifuging the conditioned media at 5000×g for 20 min.

The purification procedure was carried out using an AKTA (Amersham GE Healthcare, Upsala, Sweden) and purified. The purification was completed using a 5 mL Poros Protein A chromatography column (Applied Biosystems, Foster City, Calif.) equilibrated with 50 mM sodium acetate, 150 mM NaCl, pH 6.5. After washing, the anti-TR antibody was recovered by eluting the column with a gradient to a buffer containing 50 mM sodium acetate, 150 mM NaCl, pH 3 over 5 min. The antibody containing eluant was collected in a pool corresponding to the increase in OD₂₈₀. The pH of the glycine eluate pool was raised to pH 5 using 1 M sodium hydroxide. 1.9 mg of antibody was recovered per liter of conditioned media.

The samples were analyzed by SDS-PAGE using 4-12% pre-cast Bis-Tris gels (Invitrogen, Carlsbad, Calif.), employing Invitrogen's NuPAGE MES running buffer. Samples were made up in NuPAGE (Carlsbad, Calif.) sample preparation buffer. For reducing conditions NuPAGE sample reducing agent was added.

Proteins were transferred from a SDS-PAGE gel to a nitrocellulose membrane for identification by Western blotting with an HRP conjugated anti-mouse IgG antibody (Sigma, St. Louis, Mo.) and visualized using chemiluminescent substrate (ECL, Amersham GE Healthcare, Piscataway, N.J.).

The protein concentration was determined by the method of Bradford using reagents from Pierce Biotechnology, Rockford, Ill. The light scattering of the protein was identified in a ultraviolet spectrophotometer (Hewlett Packard, Palo Alto, Calif.) using a complete scan from 200-600 nm. A total of 2 mg AT-19 IgG was produced and purified and shown to migrate as a single peak on a gel exclusion column.

9. Secondary Antibody Characterization

Purified AT-19 Fab was characterized by ELISA and ICELISA on recombinant protein (direct) and on adherent cells expressing TR. Purified AT-19 IgG was characterized by ELISA and FACS. Cell ELISA and ICELISA were performed at 4° C. Plates were blocked for an hour in blocking buffer (5% Milk-PBS for direct assays, 2% BSA, 10% FBS-PBS for cell assays) prior to addition of the primary antibodies.

Fab ELISA: Antibodies were prepared at the desired concentration in 2% BSA and 100 μL was transferred to the ELISA plate. Incubations were for 1 hour. The ELISA plate was washed three times with wash buffer (0.05% Tween PBS for ELISA/ICELISA on recombinant protein and cold PBS with calcium and magnesium for cell ELISA/ICELISA). The secondary antibody (anti-Etag HRP, 1:5000) was then added for 30 minutes followed by 3 washed in wash buffer. The plates were were then developed with 50 μL per well Amplex red reagent as described previously.

Fab ICELISA: Antibodies were prepared at the desired concentration in 2% BSA, 1:5000 anti-Etag HRP and incubated on ice for 1 hour. 100 μL was then transferred to the ELISA plate. Incubation was for 1 hour. The plate was washed three times in washing buffer and then developed with 50 μL per well Amplex red reagent as described previously.

IgG ELISA: Antibodies were prepared at the desired concentration in 2% BSA. 100 μL was transferred to the ELISA plate. Incubation was for 1 hour. The plate was washed three times in washing buffer. 100 μL anti-mouse-IgG antibody-HRP (Sigma, St. Louis, Mo., 1:1000 in 2% BSA) was then added for 30 minutes. This was followed by three washes with washing buffer. The plates were then developed with 50 μL per well Amplex red reagent as described previously.

10. Flow Cytometry (FACS) of Anti-TR IgG on TR-Expressing Cells

Cells were detached with Cell Dissociation Buffer (GIBCO Invitrogen, Carlsbad, Calif.) and collected by centrifugation of 3000 rpm at 4° C. for 5 min. 2×10⁵ cells in 100 μl PBS buffer were incubated for 1 h at 4° C. with anti-TR antibodies at a range of concentrations. Excess antibody was washed away with 1 ml PBS followed by centrifugation, and cells were resuspended and incubated for 45 min at 4° C. in PBS 100 μl containing anti-mouse IgG-FITC (10 μg/mL, Vector Labs Inc.) as the second antibody. Excess antibody was washed away with 1 mL PBS and by centrifugation. Cells were resuspended in 200 μL PBS containing 5 μL propidium iodide (1 μg/ml). Fluorescence intensity was measured on a FACS Calibur (Becton Dickinson). Experiments were performed in triplicate. See FIGS. 5A-B.

11. Internalization Assays and Confoncal Microscopy

Cells were grown in RPMI-1640 medium (GIBCO Invitrogen, Carlsbad, Calif.) with 10% FBS on 8-well glass chamber-slides (Nalgene Nunc International, Naperville, Ill.) overnight. On the next day growth medium was replaced with medium containing primary antibody AT-19 (2 μg/well) at 4° C. for 2 h. Cells were washed three times with cold PBS. The secondary antibody, AlexaFluor-488 goat anti-Human IgG (H+L) antibody (Molecular Probes, Eugene, Oreg.), was then added at a dilution of 1:500 and incubated at 4° C. or 37° C. for 3 hr followed by three washes in cold PBS. Plasma membranes were counter-stained by Wheat Germ Agglutinin-Alexa Fluor 594 Conjugate (Molecular Probes, Eugene, Oreg.). Cells were washed three times with PBS, fixed using 5% Paraformaldehyde in PBS for 10 min at room temperature and washed two additional times using PBS. Slides were coverslipped using Fluorescent Mounting Medium (DakoCytomation, Carpinteria, Calif.).

Confocal images of the double-labelled cells were captured using a LSM5 Pascal confocal laser scanning microscope (Carl Zeiss AG, Jena, Germany) equipped with a PlanNeofluar 63×/1.25 oil objective (Carl Zeiss AG, Jena, Germany). Slides were scanned using the multi track mode (red and green channel) to avoid bleed through. Images represent a stack of twenty focal planes (1 μm apart). Single frames from representative stacks were exported into Adobe Photoshop 7.0 and saved as jpeg files.

AT-19 IgG is shown to bind specifically to TR-expressing cells by immunofluorescence. No fluorescent staining is observed for TR-negative CHOK1 cells at either 4° C. or at 37° C. Phase contrast images indicated the presence of TR-negative cells in the frame. At 4° C., AT-19 IgG is only present on the surface of the TR-positive cells. Incubation of TR-positive cells at 37° C. with AT-19 results in internalization of the Alexa 488-labeled AT-19 surface protein into vesicular cytoplasmic structures. The plasma membrane is visualized with WGA-Alexa-594. The images demonstrate that AT-19 IgG can internalize into TR over-expressing cells through binding to the TR receptor.

12. Evaluation of Panning Success

Three rounds of panning were completed using the multivalent scFv phage display library. In order to verify the success of the selection strategy the phage titers of the input, the last wash before phage elution, and the eluted phage from each round of panning were compared. Input titers are in the expected range and indicate a successful panning strategy. The input titer for round 1 is the phage concentration of the library itself. The input titers for rounds 2 and 3 are the titers of the amplified phage eluted from the previous round of panning. Elution titers represent phage released from the cell-surface after several wash steps prior to amplification. The increase in the titer of eluted phage of at least one log per round of panning indicates that a successful selection strategy has amplified specific clones at each round, thus leading to higher numbers of eluted phage in the next round of selection. Also, the comparison of the titer of the eluted phage with the titer of the last wash confirms the success of the experiment. Specifically, the increasingly stringent washing steps resulted in a substantially lower titer in the last wash as compared to the eluted phage and indicate that the eluted phage are likely specific for the target.

The insert frequency in the third round of panning was evaluated by a restriction digest (NcoI-NotI) of 20 random clones. 100% of clones were shown to contain scFv insert by this method, again indicating that non-binding, insert-less phage were removed successfully during the stringent washes.

13. Primary Screening and Sequence Analysis of Polyclonal scFv from Round 3

A total of 616 single clones from the third round phage antibody pool were screened by the reverse-capture ICELISA and by differential live cell ELISA. The primary screen yielded nineteen wells positive in both assays. These were subsequently sequenced and a single nucleic acid sequence (SEQ ID NO:2) was determined (see FIG. 7).

14. Characterization of AT19 scFv and IgG: Influence of Valency

Pure monovalent AT-19 scFv was tested for binding to native TR in a cell based ELISA. Monomeric AT-19 scFv failed to bind to the native receptor on cells. However, when the AT-19 scFv monomer is dimerized by preincubation with anti-Etag-HRP mouse IgG prior to incubation with the cells in an immuno-complex ELISA (ICELISA) format, binding to the native receptor was observed (FIG. 1, panels A and B). The binding avidity could not be quantified by ICELISA since the exact concentration of the (scFv)₂/IgG complex is unkown. However, these assay results further highlight that bivalency of AT-19 is required for binding the native receptor, that AT-19 is inactive as a monomer, and suggest the potency of the dimeric form.

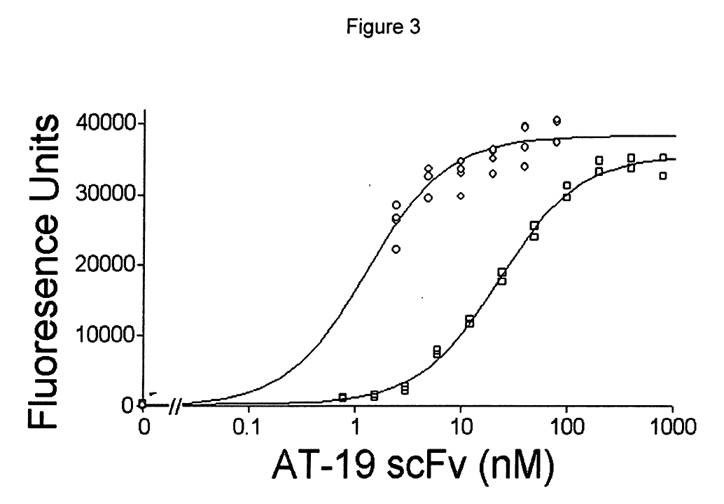

In a direct ELISA, AT-19 scFv does appear to bind to the recombinant protein as a monomer although significantly weaker than the dimer. AT-19 scFv exhibits an EC₅₀ of 22 nM in a direct ELISA. Dimerization of AT-19 in an ICELISA improves this EC₅₀ at least 20-fold to 1.3 nM demonstrating a substantial avidity effect even to the recombinant protein. (see FIG. 3)

When AT-19 is converted to the IgG the full avidity effect is observed. Dimerization of AT-19 scFv in the live cell ICELISA yields an EC₅₀ of 8.5 nM for binding to the native receptor. Again, the exact concentration of dimerized anti-TR scFv present as the desired ternary complex of (scFv)₂/IgG, is not determined. Titration of the completely dimerized format of AT-19 IgG in a cell ELISA yields an EC₅₀ of 0.44 nM (FIG. 4).

Binding of AT-19 IgG to PC3 cells expressing TR was compared to mouse anti-TR IgG 2H8 by FACS. 2H8 is a murine IgG1 monoclonal antibody that reacts with the follistatin domains of the extra-cellular region of human TR protein (Afar et al., Mol. Cancer Ther., 3:921, 2004). It is used throughout as a positive control antibody.

The observed EC₅₀ for 2H8 was 0.67 nM (FIG. 5B), a value that is very comparable to the Biacore measured affinity of 1 nM reported elsewhere (Uchida et al., Biochem. Biophys. Res. Comm. 266:593, 1999). The observed EC₅₀ for AT-19 IgG was 0.17 nM by titration and analysis by FACS (FIG. 5A). No detectable binding of the scFv monomer was observed by FACS even at concentrations as high as 2 μg/mL (80 nM, data not shown). Therefore, AT-19 exhibits an avidity effect when binding to the native receptor that cannot be quantitated exactly with by this analysis but that appears to much greater than a 20-fold effect.

All publications and patents mentioned in the above specification are herein incorporated by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An isolated human antibody, or antigen-binding antibody fragment thereof, that specifically binds to an epitope present in a tomoregulin (TR) polypeptide of SEQ ID NO:1.

2. The antibody of claim 1, wherein the antibody, or antigen binding fragment thereof, is internalized following binding to a TR expressing cell.

3. The antibody of claim 1, wherein the antibody comprises a heavy chain variable region having CDR1, CDR2 and CDR3 regions comprising the sequences set forth in SEQ ID NOS:7, 8 and 9, respectively.

4. The antibody of claim 1, wherein the antibody comprises a light chain variable region having CDR1, CDR2 and CDR3 regions comprising the sequences set forth in SEQ ID NOS:13, 14 and 15, respectively.

5. The antibody of claim 1, wherein the antibody comprises a heavy chain variable region having CDR1, CDR2 and CDR3 regions with sequences set forth in SEQ ID NOS: 7, 8 and 9, respectively, and a light chain variable region having CDR1, CDR2 and CDR3 regions with sequences set forth in SEQ ID NOS:13, 14 and 15, respectively.

6. The antibody of claim 1, wherein the antibody comprises a heavy chain variable region comprising an amino acid sequence having at least 95% sequence identity with SEQ ID NO:3.

7. The antibody of claim 1, wherein the antibody comprises a light chain variable region comprising an amino acid sequence having at least 95% sequence identity with SEQ ID NO:4.

8. The antibody of claim 1, comprising a heavy chain variable region having the amino acid sequence set forth in SEQ ID NO:3 and a light chain variable region having the amino acid sequence set forth in SEQ ID NO:4.

9. The antibody of claim 1, wherein the antibody comprises a heavy chain variable region encoded by a nucleotide sequence of SEQ ID NO:5.

10. The antibody of claim 1, wherein the antibody comprises a light chain variable region encoded by the nucleotide sequence of SEQ ID NO:6.

11. The antibody of claim 1, wherein the antibody comprises the amino acid sequence of SEQ ID NO:1.

12. The antibody of claim 1, wherein the antibody is encoded by the nucleic acid sequence of SEQ ID NO:2.

13. The antibody of claim 1, wherein the antibody fragment is selected from a group of fragments consisting of Fv, F(ab′), F(ab′)2, scFv, minibody and diabody.

14. An immunoconjugate comprising the human monoclonal antibody or antibody fragment of claim 1, wherein the antibody or antibody fragment is conjugated to a molecule which is a therapeutic agent or a detectable marker.

15. The immunoconjugate of claim 14, wherein the therapeutic agent is a cytotoxic agent.

16. The immunoconjugate of claim 15, wherein the cytotoxic agent is selected from a group consisting of ricin, doxorubicin, daunorubicin, paclitaxel (TAXOL™), ethidium bromide, mitomycin, etoposide, tenoposide, vincristine, vinblastine, colchicine, dihydroxy anthracin dione, actinomycin D, diphteria toxin, Pseudomonas exotoxin (PE) A, epothilones, monomethyl auristatin (MMAE), PE40, ricin, abrin, glucocorticoid and radioisotopes.

17. The immunoconjugate of claim 14, wherein the cytotoxic agent is a radioisotope and is selected from a group consisting of ⁴⁶Sc, ⁴⁷Sc, ⁴⁸Sc, ⁷²Ga, ⁷³Ga, ⁹⁰Y, ⁶⁷Cu, ¹⁰⁹Pd, ¹¹Ag, ¹⁴⁹Pm, ¹⁵³Sm, ¹⁶⁶Ho, ¹⁷⁷Lu, ¹⁸⁶Re, ¹⁸⁸Re, ²¹¹At, ²¹¹Bi, ²¹²Bi, ²¹³Bi and ²¹⁴Bi.

18. The immunoconjugate of claim 14, wherein the detectable marker is a radiolabel, an enzyme, a chromophore, or a fluorescer.

19. A method for selectively destroying a cancer cell expressing a human tomoregulin polypeptide, comprising reacting the immunoconjugate of claim 14 with the cell such that the cell is destroyed.

20. The method of claim 19, wherein the cancer cell is a prostate cancer cell.

21. A method for treating a cancer in a subject, wherein the method comprises administering to the patient a therapeutically effective amount of the immunoconjugate of claim 14.

22. The method of claim 21, wherein the cancer is prostate cancer.

23. A method of detecting cancer in a subject, wherein the method comprises:
(a) administering to the subject the immunoconjugate of claim 18;
(b) detecting the binding of the immunoconjugate within the subject; and
(c) determining if the level of binding of the immunoconjugate in the subject is increased as compared with the level of binding detected in disease-free control subjects.

24. A method for isolating a human antibody against an antigen (or a cell-surface antigen), wherein the method comprises the steps of
a) generating a multivalent scFv phage display library using rescuing with hyperphage;
b) selecting the scFv phage from the phage display library by alternating the cell-surface selection used during panning; and
c) screening the selected scFv phage with an immunocomplex ELISA format.
